(19) United States
(12) Patent Application Publication
(10) Pub. No.: US 2024/0424192 A1
(43) Pub. Date: Dec. 26, 2024

(54) MOBILITY UNIT FOR NEGATIVE-PRESSURE TREATMENT

(71) Applicant: KCI Manufacturing Unlimited Company, Westmeath (IE)

(72) Inventors: Shannon C. INGRAM, San Antonio, TX (US); Kevin HIGLEY, San Antonio, TX (US)

(21) Appl. No.: 18/274,104

(22) PCT Filed: Jan. 7, 2022
(86) PCT No.: PCT/IB2022/050113

§ 371 Date: Jul. 25, 2023

Related U.S. Application Data
(60) Provisional application No. 63/141,720, filed on Jan. 26, 2021.

(51) Int. Cl. A61M 1/00; A61M 39/24

(52) U.S. Cl. CPC A61M 1/966; A61M 1/81; A61M 39/24

(57) ABSTRACT

An apparatus for negative-pressure treatment of a tissue site may comprise a fluid reservoir having a variable volume; a first fluid passage fluidly coupled to the fluid reservoir; a first check valve configured to prevent egress through the first fluid passage; a second fluid passage fluidly coupled to the fluid reservoir; and a second check valve configured to prevent ingress through the second fluid passage. In some embodiments, the first check valve and the second check valve may be configured to open if downstream negative pressure is greater than upstream negative pressure. The first fluid passage may be configured to be coupled to a dressing or tissue interface, and the second fluid passage may be configured to be coupled to a negative-pressure source. The apparatus may provide a mobile, re-usable, self-emptying, self-charging source of negative pressure that can manage fluid and temporary or transitional negative-pressure treatment.

### CROSS-REFERENCE TO RELATED APPLICATIONS

This application is a U.S. National Stage entry of International Patent Application No. PCT/IB2022/050113, filed on Jan. 7, 2022, which claims the benefit of priority to U.S. Provisional Application No. 63/141,720, filed on Jan. 26, 2021, which is incorporated herein by reference in its entirety.

### TECHNICAL FIELD

The invention set forth in the appended claims relates generally to tissue treatment systems and more particularly, but without limitation, to apparatuses, systems, and methods for treating tissue with negative pressure.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for treating a tissue site in a negative-pressure therapy environment are set forth in the appended claims. Illustrative embodiments are also provided to enable a person skilled in the art to make and use the claimed subject matter.

In some embodiments, an apparatus for managing fluid for negative-pressure treatment of a tissue site may comprise a fluid reservoir or chamber having a variable volume; a first fluid passage fluidly coupled to the fluid reservoir; a first check valve configured to prevent egress through the first fluid passage; a second fluid passage fluidly coupled to the fluid reservoir; and a second check valve configured to prevent ingress through the second fluid passage. In some embodiments, the first check valve and the second check valve may be configured to open if downstream negative pressure is greater than upstream negative pressure. The first fluid passage may be configured to be coupled to a dressing or tissue interface, and the second fluid passage may be configured to be coupled to a negative-pressure source. The fluid reservoir may be configured to contract if negative pressure increases in the fluid reservoir and to expand if negative pressure decreases in the fluid reservoir. The apparatus may additionally comprise a third fluid passage in some embodiments, and the third fluid passage may comprise a first end fluidly coupled to the first fluid passage and a second end fluidly coupled to the second fluid passage.

Some example embodiments may comprise a system for treating a tissue site with negative pressure, the system comprising a housing and a piston defining a fluid chamber; a first fluid passage fluidly coupled to the fluid chamber and configured to be fluidly coupled to a dressing; a first check valve configured to prevent egress from the fluid chamber through the first fluid passage; a second fluid passage fluidly coupled to the fluid chamber; a second check valve configured to prevent ingress to the fluid chamber through the second fluid passage; and a negative-pressure source configured to be coupled to the fluid chamber through the second fluid passage. The fluid chamber may be configured to contract if negative pressure increases in the fluid chamber and to expand if negative pressure decreases in the fluid chamber. Some embodiments may additionally comprise a pressure sensor, a third fluid passage configured to couple the pressure sensor to the dressing, and a controller configured to operate the negative-pressure source based on input from the pressure sensor. The second check valve can be configured to maintain a therapeutic level of negative pressure in the fluid chamber if the negative-pressure source is separated from the fluid chamber.

Some embodiments may comprise methods of treating a tissue site with negative pressure. For example, some methods may comprise disposing a dressing adjacent to the tissue site and providing a fluid management unit. The fluid management unit can comprise a housing, a piston defining a fluid chamber in the housing, a first fluid passage fluidly coupled to the fluid chamber, a first check valve configured to prevent egress from the fluid chamber through the first fluid passage, a second fluid passage fluidly coupled to the fluid chamber, and a second check valve configured to prevent ingress to the fluid chamber through the second fluid passage. The method can further include fluidly coupling the fluid chamber to the dressing through the first check valve, fluidly coupling a negative-pressure source to the fluid chamber through the second check valve, and applying a therapeutic level of negative-pressure from the negative-pressure source to the fluid chamber. Some methods may additionally comprise separating the negative-pressure source from the fluid chamber, whereby the second check valve closes to maintain a therapeutic level of negative pressure in the fluid chamber and the fluid chamber receives fluid from dressing through the first check valve.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a functional block diagram of an example embodiment of a therapy system that can provide negative-pressure treatment in accordance with this specification;

FIG. 2 is a schematic diagram of an example of an auxiliary therapy unit that may be associated with some embodiments of the therapy system of FIG. 1;

FIG. 3 is a schematic diagram of the auxiliary therapy unit of FIG. 2, illustrating additional details that may be associated with some embodiments;

FIG. 4 is a schematic diagram of an example of the therapy system with the auxiliary therapy unit of FIG. 3;

FIG. 5 is a schematic diagram of an example embodiment of the therapy system of FIG. 1, illustrating additional details that may be associated with some embodiments;

FIG. 6 is a schematic diagram of an example embodiment of the therapy system of FIG. 1, illustrating additional details that may be associated with some embodiments;

FIG. 7 is a schematic diagram of an example embodiment of the therapy system of FIG. 1, illustrating additional details that may be associated with some embodiments; and

FIG. 8 is a schematic diagram of an example embodiment of the therapy system of FIG. 1, illustrating additional details that may be associated with some embodiments.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive treatment. However, as should be recognized by those skilled in the art, this frame of reference is merely a descriptive expedient rather than a strict prescription.

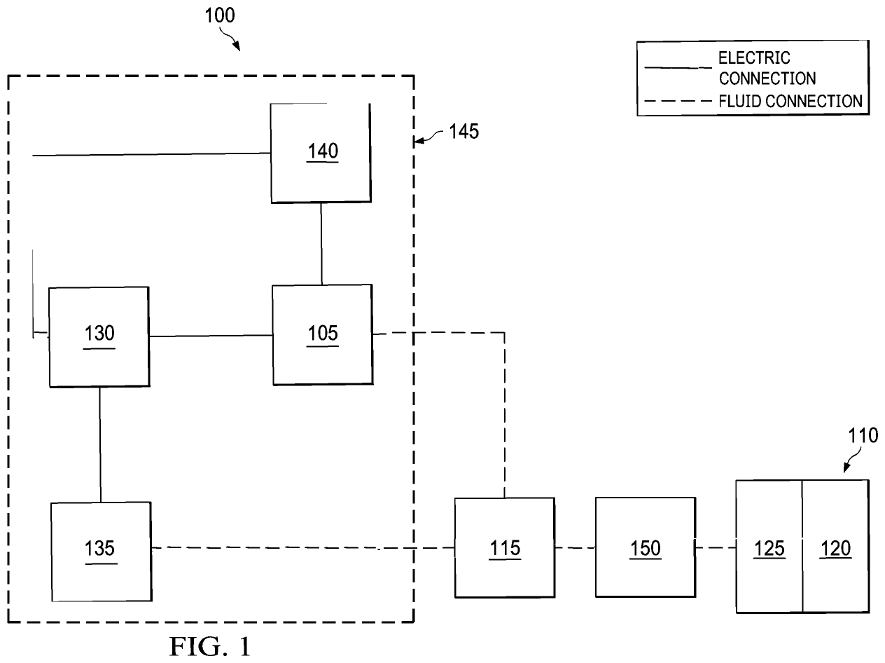

FIG. 1 is a simplified functional block diagram of an example embodiment of a therapy system 100 that can provide negative-pressure therapy to a tissue site in accordance with this specification. The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit. In the example of FIG. 1, the negative-pressure source 105, the controller 130, the first sensor 135, and the second sensor 140 are combined in a primary therapy unit 145.

The therapy system 100 may additionally comprise an auxiliary therapy unit 150, which may be configured to provide transitional or temporary therapy. For example, the auxiliary therapy unit 150 may be fluidly coupled between the dressing 110 and the primary therapy unit 145. In some embodiments, the auxiliary therapy unit 150 may be fluidly coupled between the dressing 110 and the container 115. It may be beneficial or advantageous to disconnect the dressing 110 from the primary therapy unit 145 to increase mobility. For example, the dressing 110 and the auxiliary therapy unit 150 may be disconnected from the primary therapy unit 145 and the auxiliary therapy unit 150 can continue to provide therapeutic levels of therapy and exudate management while disconnected.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols, such as polyester or polyether, isocyanate, such as toluene diisocyanate, and polymerization modifiers, such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and caprolactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours (g/m²/24 hours) in some embodiments, measured using an upright cup technique according to ASTM E96/E96M Upright Cup Method at 38° C. and 10% relative humidity (RH). In some embodiments, an MVTR up to 5,000 g/m²/24 hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polyamide copolymers. Such materials are commercially available as, for example, Tegaderm® drape, commercially available from 3M Company, Minneapolis, Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Transcontinental Advanced Coating, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The fluid mechanics of using a negative-pressure source to reduce pressure in another component or location, such as within a sealed therapeutic environment, can be mathematically complex. However, the basic principles of fluid mechanics applicable to negative-pressure therapy are generally well-known to those skilled in the art, and the process of reducing pressure may be described illustratively herein as “delivering,” “distributing,” or “generating” negative pressure, for example.

In general, exudate and other fluid flow toward lower pressure along a fluid path. Thus, the term “downstream” typically implies something in a fluid path relatively closer to a source of negative pressure or further away from a source of positive pressure. Conversely, the term “upstream” implies something relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as a limiting convention.

Negative pressure applied across the tissue site through the tissue interface 120 in the sealed therapeutic environment can induce macro-strain and micro-strain in the tissue site. Negative pressure can also remove exudate and other fluid from a tissue site, which can be collected in container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

In some embodiments, the controller 130 may have a continuous pressure mode, in which the negative-pressure source 105 is operated to provide a constant target negative pressure for the duration of treatment or until manually deactivated. Additionally or alternatively, the controller may have an intermittent pressure mode. For example, the controller 130 can operate the negative-pressure source 105 to cycle between a target pressure and atmospheric pressure. For example, the target pressure may be set at a value of −125 mmHg for a specified period of time (e.g., 5 minutes), followed by a specified period of time (e.g., 2 minutes) of deactivation. The cycle can be repeated by activating the negative-pressure source 105, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time may be a value substantially equal to the initial rise time.

In some example dynamic pressure control modes, the target pressure can vary with time. For example, the target pressure may vary in the form of a triangular waveform, varying between a negative pressure of 50 and 125 mmHg with a rise time set at a rate of +25 mmHg/min and a descent time set at −25 mmHg/min. In other embodiments of the therapy system 100, the triangular waveform may vary between negative pressure of 25 and 125 mmHg with a rise time set at a rate of +30 mmHg/min and a descent time set at −30 mmHg/min.

In some embodiments, the controller 130 may control or determine a variable target pressure in a dynamic pressure mode, and the variable target pressure may vary between a maximum and minimum pressure value that may be set as an input prescribed by an operator as the range of desired negative pressure. The variable target pressure may also be processed and controlled by the controller 130, which can vary the target pressure according to a predetermined waveform, such as a triangular waveform, a sine waveform, or a saw-tooth waveform. In some embodiments, the waveform may be set by an operator as the predetermined or time-varying negative pressure desired for therapy.

Some therapy systems may be portable and operate independently from an electrical connection or another electrically powered device to provide mobility to users. In some cases, a portable therapy unit can only be used once and must be replaced. For example, the portable therapy unit may be capable of only being charged once to deliver negative pressure to a dressing, such as the dressing 110. Further, some portable therapy units can store fluid within the portable therapy unit. If the portable therapy unit becomes full, it must be discarded and replaced. The need to regularly replace the portable therapy unit may complicate the provision of therapy for some users.

These limitations and others can be addressed by the therapy system 100, which can include a reusable mobility pump, such as the auxiliary therapy unit 150. The auxiliary therapy unit 150 can be disconnected from a primary therapy system, such as the primary therapy unit 145, for an extended period of time while providing portable, mechanically-powered negative pressure. If reconnected to the primary therapy unit 145, the auxiliary therapy unit 150 can be emptied by the primary therapy unit 145 automatically, without further intervention, and the auxiliary therapy unit 150 can be recharged for future portability.

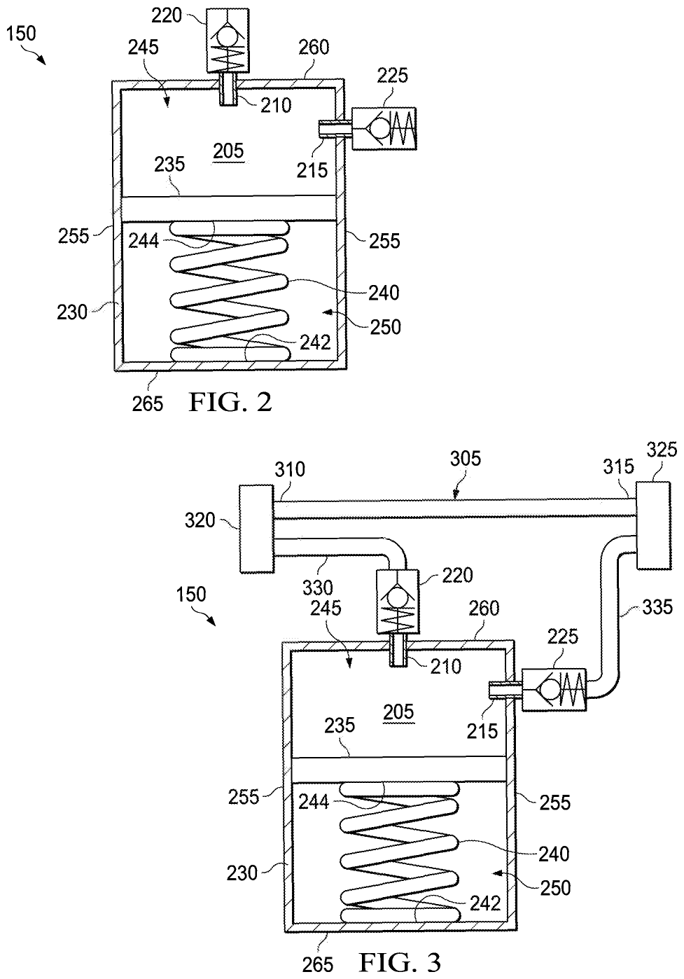

FIG. 2 is a schematic diagram of an example of the auxiliary therapy unit 150, illustrating additional details that may be associated with some embodiments. In some embodiments, the auxiliary therapy unit 150 include a housing 230. The housing 230 may form a portion of an exterior of the auxiliary therapy unit 150. In some embodiments, the housing 230 may comprise sidewalls 255, a first end wall 260, and a second end wall 265. The first end wall 260 and the second end wall 265 may be coupled to the sidewalls 255 to form the housing 230. The second end wall 265 may be coupled to the sidewalls 255 opposite the first end wall 260. The sidewalls 255, the first end wall 260, and the second end wall 265 may form an interior of the housing 230. In some embodiments, the interior formed by the housing 230, the sidewalls 255, the first end wall 260, and the second end wall 265 may be fluidly isolated from the ambient environment.

In some embodiments, the auxiliary therapy unit 150 may include a fluid reservoir 205, a first fluid passage 210 fluidly coupled to the fluid reservoir 205, and a second fluid passage 215 fluidly coupled to the fluid reservoir 205. The fluid reservoir 205 may be defined by the interior of the housing 230. In some embodiments, a first check valve 220 may be fluidly coupled to the first fluid passage 210 and a second check valve 225 may be fluidly coupled to the second fluid passage 215. A check valve may be a valve that permits one-way flow of fluid through the valve. For example, the first check valve 220 may be configured to prevent egress from the fluid reservoir 205 through the first fluid passage 210. Exemplary check valves, such as the first check valve 220 and the second check valve 225, may include ball check valves, diaphragm check valves, swing check valves, stop-check valves, duckbill valves, lift valves, butterfly valves, or tilting disc valves. In some embodiments, the first check valve 220 may be normally closed, and may be configured to allow ingress through the first fluid passage 210 if negative pressure in the fluid reservoir 205 is greater than zero. For example, if a negative pressure is developed in the fluid reservoir 205, the first check valve 220 may open and fluid may flow through the first fluid passage 210 into the fluid reservoir 205. In some embodiments, the first check valve 220 may be configured to allow ingress through the first fluid passage 210 if the pressure differential between the upstream pressure and the downstream pressure is in a range of about 20 mm Hg to about 100 mm Hg. In some embodiments, the first check valve 220 may be configured to allow ingress through the first fluid passage 210 if the pressure differential is about 50 mm Hg.

In some embodiments, the first check valve 220 may have a cracking pressure. The cracking pressure may be the minimum pressure differential across a check valve between downstream pressure and upstream pressure at which the check valve will open and allow fluid to move through. For example, if the first check valve 220 has a cracking pressure of 20 mm Hg, the first check valve 220 is configured to open if the downstream negative pressure is at least 20 mm Hg greater than the upstream negative pressure. In some embodiments, the first check valve 220 may have a cracking pressure in a range of about 10 mm Hg to about 60 mm Hg. In some embodiments, a cracking pressure of at least 50 mm Hg may be particularly advantageous for the first check valve 220.

The second check valve 225 may be configured to prevent ingress to the fluid reservoir 205 through the second fluid passage 215. In some embodiments, the second check valve 225 may be normally closed, and may be configured to allow egress through the second fluid passage 215 if negative pressure from the negative-pressure source 105 is greater than negative pressure in the fluid reservoir 205. For example, if the negative-pressure source 105 generates a negative pressure greater than the negative pressure in the fluid reservoir 205, the second check valve 225 may open and fluid may flow through the second fluid passage 215 from the fluid reservoir 205 toward the negative-pressure source 105. In some embodiments, the second check valve 225 may be configured to allow egress through the second fluid passage 215 if the pressure differential between the upstream pressure and the downstream pressure is in a range of about 30 mm Hg to about 110 mm Hg. In some embodiments, the second check valve 225 may be configured to allow egress through the second fluid passage 215 if the pressure differential is about 50 mm Hg.

In some embodiments, the second check valve 225 may be configured to open if downstream negative pressure is greater than upstream negative pressure. For example, if the second check valve 225 has a cracking pressure of 20 mm Hg, the second check valve 225 is configured to open if the downstream negative pressure is at least 20 mm Hg greater than the upstream negative pressure. In some embodiments, the first check valve 220 and the second check valve 225 may have the same cracking pressure. For example, the second check valve 225 may have a cracking pressure in a range of about 10 mm Hg to about 60 mm Hg. In some embodiments, a cracking pressure of at least 50 mm Hg may be particularly advantageous for the second check valve 225.

In some embodiments, the auxiliary therapy unit 150 may include a piston 235. The piston 235 may be positioned within the fluid reservoir 205 of the housing 230 and form a first chamber 245 and a second chamber 250 within the fluid reservoir 205. The first chamber 245 may extend from the piston 235 to the first end wall 260. The second chamber 250 may extend from the piston 235 to the second end wall 265. In some embodiments, the first chamber 245 may be open to the ambient environment and the second chamber 250 may be sealed from the ambient environment. In some embodiments, the piston 235 may be moveable within the housing 230 to vary respective volumes of the first chamber 245 and the second chamber 250. For example, the volume of the first chamber 245 and the second chamber 250 may vary as negative pressure in the first chamber 245 varies.

In some embodiments, the auxiliary therapy unit 150 may include a biasing element, such as a piston spring 240, to bias the piston 235. The piston spring 240 may be disposed in the second chamber 250 of the fluid reservoir 205 between the piston 235 and the second end wall 265. For example, the piston spring 240 may have a first end 242 coupled to the second end wall 265 of the housing 230 and a second end 244 coupled to the piston 235. In some embodiments, the piston spring 240 may be a coiled spring. The coiled spring may be constructed from a rolled ribbon of spring steel or similar having a first end, such as the first end 242, coupled to the second end wall 265 and a second end, such as the second end 244, coupled to the piston 235. The second end 244 of the piston spring 240 can be extended to generate a spring force. In some embodiments, other springs may be used. A spring constant of the piston spring 240 may be selected for a desired therapeutic pressure of the first chamber 245.

In some embodiments, the piston spring 240 may have an unloaded position and a loaded position. Generally, if no external force is acting on the piston spring 240, the piston spring 240 is in the unloaded position. If the piston spring 240 is extended or compressed, the piston spring 240 is in the loaded position, as shown in FIG. 2. Generally, a spring may exert a reactive force in response to displacement from the unloaded positioned. The reactive force is generally proportional to the distance a spring is either compressed or extended if an external force loads the spring.

In some embodiments, negative pressure in the first chamber 245 may overcome the spring force of the piston spring 240. For example, if the primary therapy unit 145 is configured to provide negative pressure of 125 mm Hg, the negative pressure in the first chamber 245 may overcome the spring force of the piston spring 240, drawing the piston 235 toward the first end wall 260. The piston spring 240 may expand to the loaded position as a volume of the first chamber 245 decreases and a volume of the second chamber 250 increases. As negative pressure decreases in the first chamber 245, negative pressure may no longer overcome the spring force of the piston spring 240. In response, the piston spring 240 may return to the unloaded positioned and draw the piston 235 toward the second end wall 265, increasing the volume of the first chamber 245 and decreasing the volume of the second chamber 250.

In other embodiments, the piston spring 240 may be disposed in the first chamber 245. For example, the first end 242 of the piston spring 240 may be coupled to the piston 235 and the second end 244 of the piston spring 240 may be coupled to the first end wall 260. In some embodiments, an increase in negative pressure in the first chamber 245 can overcome the spring force of the piston spring 240 and the piston spring 240 may move the piston 235 toward the first end wall 260, decreasing the volume of the first chamber 245 and increasing the volume of the second chamber 250. A decrease in negative pressure in the first chamber 245 can cause the piston spring 240 to return to the unloaded position, moving the piston 235 toward the second end wall 265, which can increase the volume of the first chamber 245 and decrease the volume of the second chamber 250.

In some embodiments, the auxiliary therapy unit 150 may additionally comprise a means for limiting the movement of the piston 235. For example, the second fluid passage 215 may extend into the fluid reservoir 205, which can prevent the piston 235 and the piston spring 240 from moving past the second fluid passage 215 and blocking the first fluid passage 210 and the second fluid passage 215. In other embodiments, a pin or rod may be positioned or inserted in at least one of the sidewalls 255 of the housing 230 below the second fluid passage 215. In other embodiments, a ridge may be formed on an interior surface of at least one of the sidewalls 255 of the housing 230 below the second fluid passage 215 to prevent the piston 235 and the piston spring 240 from extending past the second fluid passage 215.

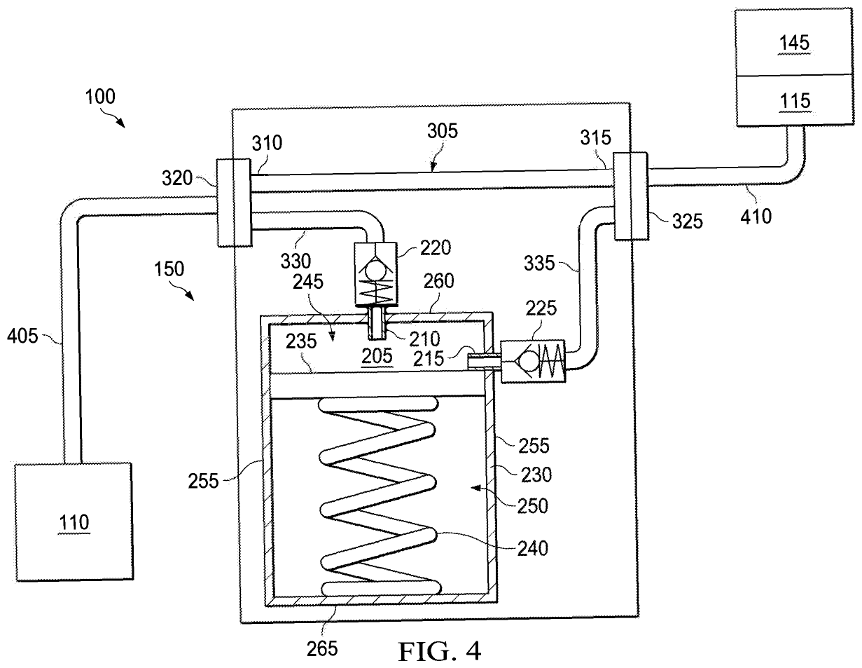

FIG. 3 is a schematic diagram of the auxiliary therapy unit 150 of FIG. 2, illustrating additional details that may be associated with some embodiments. In some embodiments, the auxiliary therapy unit 150 may comprise a third fluid passage 305. The third fluid passage 305 may comprise a first end 310 and a second end 315. The first end 310 may be fluidly coupled to the first fluid passage 210 and the second end 315 may be fluidly coupled to the second fluid passage 215.

In some embodiments, the auxiliary therapy unit 150 may comprise a first adapter 320 and a second adapter 325. The first adapter 320 may be configured to couple the first end 310 of the third fluid passage 305 to the first fluid passage 210. In some embodiments, the first adapter 320 may fluidly couple the third fluid passage 305 to another distribution component, such as a multi-lumen conduit. Similarly, the first adapter 320 may fluidly couple the first fluid passage 210 to another distribution component, such as a multi-lumen conduit. In some embodiments, the first adapter 320 may fluidly couple the first fluid passage 210 to a first lumen of a multi-lumen conduit and fluidly coupled the third fluid passage 305 to another lumen of the same multi-lumen conduit. In some embodiments, the first adapter 320 may be fluidly coupled to the first fluid passage 210 through one or more intermediate distribution components. For example, the first adapter 320 may be fluidly coupled to the first fluid passage 210 via a fluid conductor 330. In some embodiments, the fluid conductor 330 may be a single-lumen tube or conduit. In other embodiments, the fluid conductor 330 may be a multi-lumen tube or conduit.

In some embodiments, the second adapter 325 may be configured to couple the second end 315 of the third fluid passage 305 to the second fluid passage 215. In some embodiments, the second adapter 325 may fluidly couple the third fluid passage 305 to another distribution component, such as a multi-lumen conduit. Similarly, the second adapter 325 may fluidly couple the second fluid passage 215 to another distribution component, such as a multi-lumen conduit. In some embodiments, the second adapter 325 may fluidly couple the second fluid passage 215 to a first lumen of a multi-lumen conduit and fluidly coupled the third fluid passage 305 to another lumen of the same multi-lumen conduit. In some embodiments, the second adapter 325 may be fluidly coupled to the second fluid passage 215 through one or more intermediate distribution components. For example, the second adapter 325 may be fluidly coupled to the second fluid passage 215 via a fluid conductor 335. In some embodiments, the fluid conductor 335 may be a single lumen tube or conduit. In other embodiments, the fluid conductor 335 may be a multi-lumen tube or conduit.

FIG. 4 is a schematic diagram of an example of the therapy system 100 with the primary therapy unit 145 of FIG. 1 and the auxiliary therapy unit 150 of FIG. 3. In some embodiments, the dressing 110 may be fluidly coupled to the primary therapy unit 145 through the auxiliary therapy unit 150. For example, the dressing 110 may be fluidly coupled to the first fluid passage 210, which is fluidly coupled to the first chamber 245. The primary therapy unit 145 may be fluidly coupled to the first chamber 245 through the second fluid passage 215. More particularly, a negative-pressure source associated with the primary therapy unit 145, such as the negative-pressure source 105 of FIG. 1, may be fluidly coupled to the first chamber 245.

In some embodiments, the dressing 110 may be fluidly coupled to the first fluid passage 210 through one or more intermediate distribution components, such as a fluid conductor 405. For example, the first adapter 320 may be configured to receive the fluid conductor 405 and fluidly couple the fluid conductor 405 to the first fluid passage 210 through the fluid conductor 330 and the first check valve 220.

In some embodiments, the primary therapy unit 145 may be fluidly coupled to the second fluid passage 215 through one or more intermediate distribution components, such as a fluid conductor 410. For example, the second adapter 325 may be configured to receive the fluid conductor 410 and to fluidly couple the fluid conductor 410 to the second fluid passage 215 through the fluid conductor 335 and the second check valve 225. In other embodiments, the auxiliary therapy unit 150 may be configured to directly couple the second fluid passage 215 to the primary therapy unit 145. For example, the primary therapy unit 145 may have a port configured to receive the second fluid passage 215, and the auxiliary therapy unit 150 may be configured to snap onto the primary therapy unit 145.

Additionally, in some embodiments, the dressing 110 may be fluidly coupled to the primary therapy unit 145 via the fluid conductor 405, the first adapter 320, the fluid conductor 330, the auxiliary therapy unit 150, the fluid conductor 335, the second adapter 325, and the fluid conductor 410 to form a negative-pressure pathway. The negative-pressure pathway may be configured to deliver negative pressure to the dressing 110. In some embodiments, the dressing 110 may also be fluidly coupled to the primary therapy unit 145 via the fluid conductor 405, the first adapter 320, the third fluid passage 305, the second adapter 325, and the fluid conductor 410 to form a sensing pathway. More particularly, the dressing 110 may be fluidly coupled to the first sensor 135 associated with the primary therapy unit 145 via the sensing pathway. In some embodiments, the sensing pathway may be configured to measure negative pressure at the dressing 110.

In some embodiments, the first adapter 320 may be configured to receive the fluid conductor 405 and fluidly couple the fluid conductor 405 to the first fluid passage 210 and the third fluid passage 305. In some embodiments, the fluid conductor 405 and the fluid conductor 410 may each comprise a multi-lumen tube or conduit. The first adapter 320 may be configured to fluidly couple at least one lumen of the fluid conductor 405 to the first fluid passage 210 and fluidly couple at least one other lumen of the fluid conductor 405 to the first end 310 of the third fluid passage 305. The second adapter 325 may be configured to fluidly couple at least one lumen of the fluid conductor 410 to the second end 315 of the third fluid passage 305 and fluidly couple at least one other lumen of the fluid conductor 410 to the second fluid passage 215.

In operation, the dressing 110 may be disposed adjacent to a tissue site (not shown). Negative pressure from the primary therapy unit 145 can be distributed to the dressing 110 through the auxiliary therapy unit 150, and exudate may be removed from the tissue site and collected in the container 115 of the primary therapy unit 145. The container 115 may be fluidly coupled to the dressing 110. In some embodiments, the container 115 may be coupled to the primary therapy unit 145. In other embodiments, the container 115 may be housed within the primary therapy unit 145.

In some embodiments, negative pressure from the primary therapy unit 145 can open the second check valve 225, which can reduce the pressure in the first chamber 245. The negative pressure in the first chamber 245 may generate a differential pressure across the piston 235 that exerts a force on the piston 235. The force may urge the piston 235 toward the first end wall 260, extending the piston spring 240 to the loaded position. As shown in FIG. 4, the auxiliary therapy unit 150 may be in a charged position. As negative pressure is developed in the first chamber 245, drawing the piston 235 and the piston spring 240 to the loaded position, the negative pressure may open the first check valve 220. Negative pressure can then be applied to the dressing 110 from the primary therapy unit 145 through a fluid path comprising the fluid conductor 405, the first adapter 320, the fluid conductor 330, the first check valve 220, the first fluid passage 210, the first chamber 245, the second fluid passage 215, the second check valve 225, the fluid conductor 335, the second adapter 325, the fluid conductor 410, and the container 115.

Additionally, in some embodiments, the primary therapy unit 145 can measure pressure at the dressing 110 through the auxiliary therapy unit 150. For example, a sensor, such as the first sensor 135 of FIG. 1, can be contained within the primary therapy unit 145 and sample pressure through the sensing pathway comprising the fluid conductor 405, the first adapter 320, the third fluid passage 305, the second adapter 325, and the fluid conductor 410. A controller, such as the controller 130 of FIG. 1, can operate a negative-pressure source, such as the negative-pressure source 105 of FIG. 1, based on input from the first sensor 135 to provide therapeutic levels of negative pressure to a tissue site.

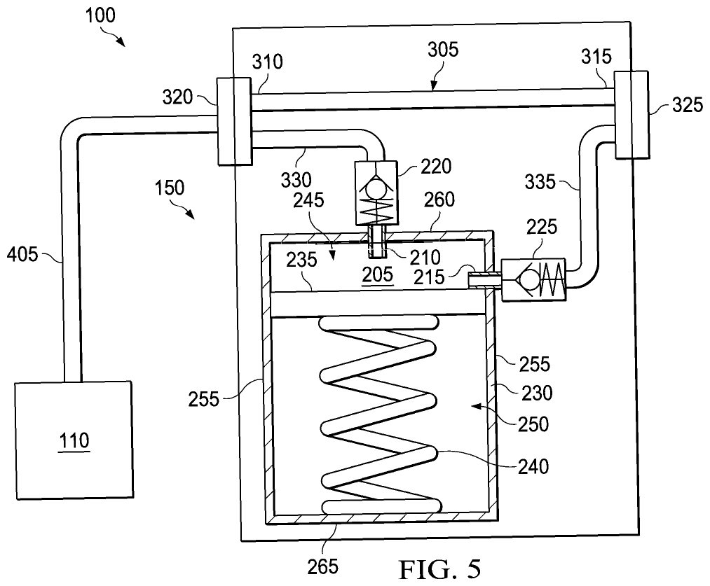

FIG. 5 is a schematic diagram of an example embodiment of the therapy system 100 of FIG. 1, illustrating additional details that may be associated with some embodiments. The auxiliary therapy unit 150 is generally configured to maintain a therapeutic level of negative pressure if the primary therapy unit 145 is disconnected. For example, if the fluid conductor 410 of FIG. 4 is separated from the second adapter 325, the second check valve 225 closes to maintain negative pressure in the first chamber 245. As negative pressure remains in the first chamber 245, the auxiliary therapy unit 150 may remain in the charged position. For example, the piston 235 may be in contact with the second fluid passage 215 and the piston spring 240 may be extended and in the loaded position. Additionally, negative pressure in the first chamber 245 can keep the first check valve 220 open, and negative pressure from the first chamber 245 can be delivered to the dressing 110 through the first fluid passage 210. In some embodiments, the second adapter 325 may include a valve fluidly coupled to the second end 315 of the third fluid passage 305. If the fluid conductor 410 is separated from the second adapter 325, the valve may close, blocking the third fluid passage 305. If the fluid conductor 410 is connected to the second adapter 325, the valve may open, opening the third fluid passage 305.

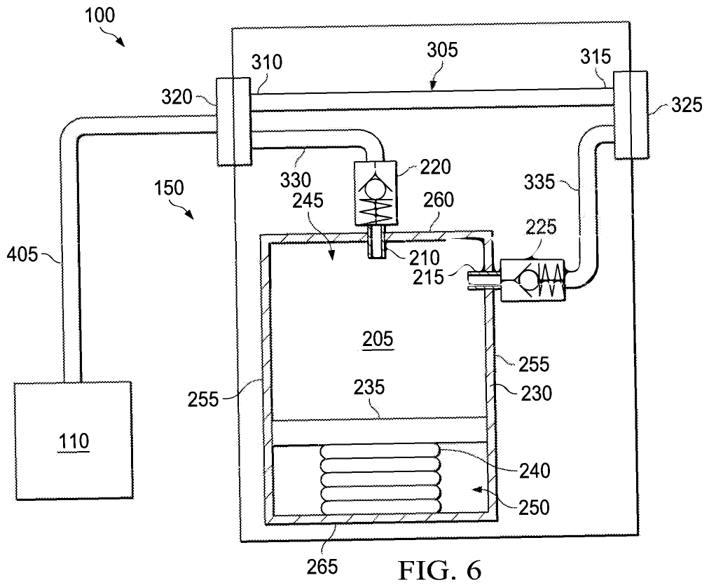

FIG. 6 is a schematic diagram of an example embodiment of the therapy system 100 of FIG. 1, illustrating additional details that may be associated with some embodiments. As shown in FIG. 6, the auxiliary therapy unit 150 is in an unloaded position. As negative pressure is delivered to the dressing 110, negative pressure in the first chamber 245 decreases. The piston spring 240 can expand the volume of the first chamber 245 and decrease the volume of the second chamber 250 to compensate for decreases in negative pressure. For example, as negative pressure in the first chamber 245 decreases, the piston spring 240 may compress and exert a force on the piston 235 that urges the piston 235 toward the second end wall 265. Exudate and other fluid can be received through the first fluid passage 210 and can be collected in the first chamber 245.

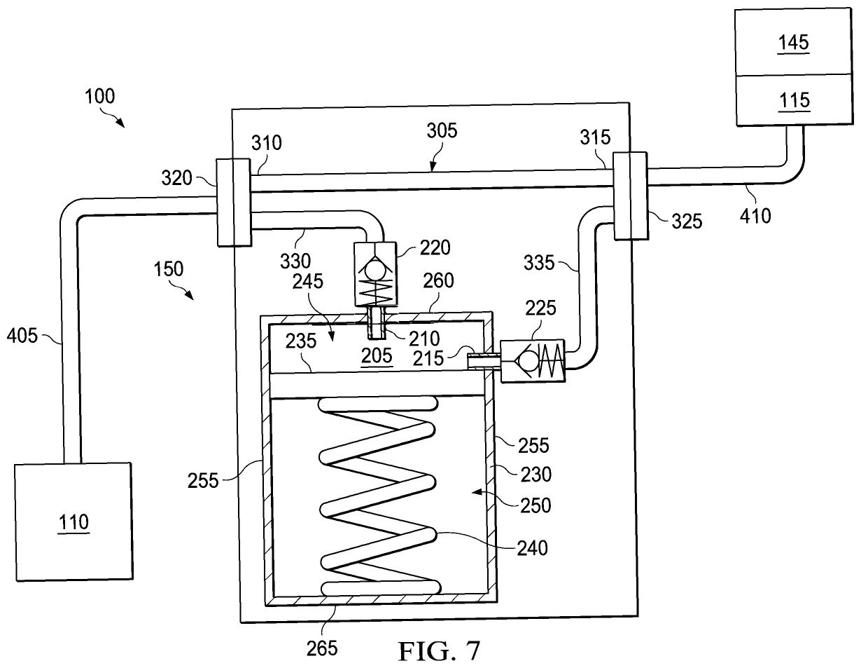

FIG. 7 is a schematic diagram of an example embodiment of the therapy system 100 of FIG. 1, illustrating additional details that may be associated with some embodiments. If the primary therapy unit 145 is re-connected to the auxiliary therapy unit 150, negative pressure from the primary therapy unit 145 can open the second check valve 225, and any exudate or other fluid in the first chamber 245 can be removed through the second fluid passage 215 and collected in the container 115. As fluids including exudates in the first chamber 245 are drawn into the container 115, negative pressure can be developed in the first chamber 245. The negative pressure in the first chamber 245 may generate a differential pressure across the piston 235 that exerts a force on the piston 235. The force may draw the piston 235 toward the first end wall 260, extending the piston spring 240 to the loaded position. Thus, as the auxiliary therapy unit 150 is drained of fluids from the tissue site, the auxiliary therapy unit 150 is recharged and returned to the charged position, as shown in FIG. 7. After the auxiliary therapy unit 150 is recharged, the primary therapy unit 145 can be disconnected once again and the auxiliary therapy unit 150 can continue delivering negative pressure to the dressing 110.

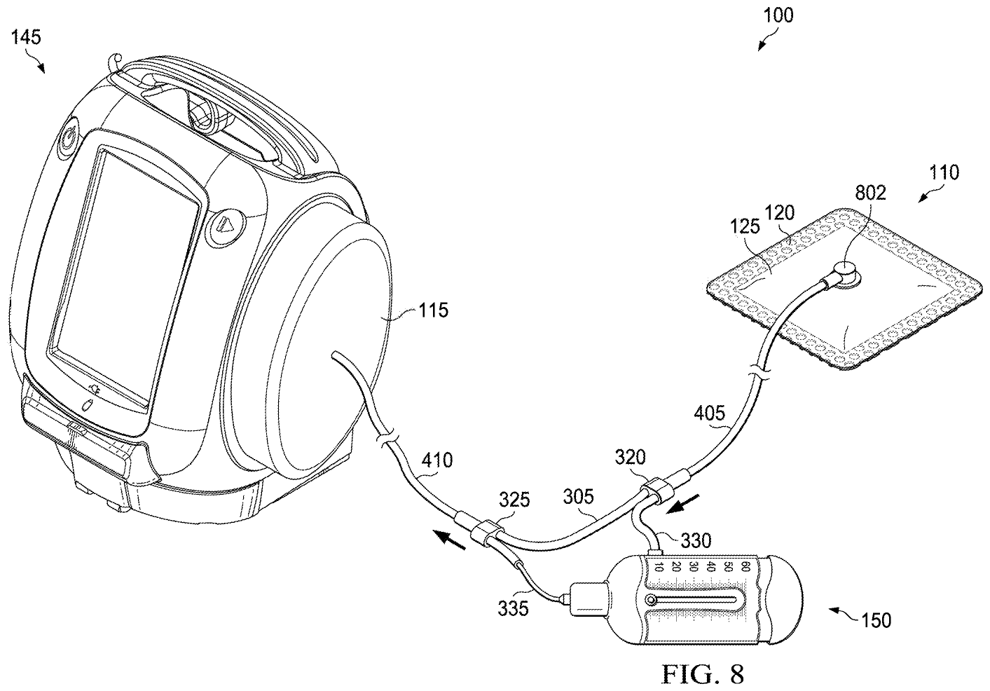

FIG. 8 is a schematic diagram of an example embodiment of the therapy system 100 of FIG. 1, illustrating additional details that may be associated with some embodiments. The therapy system 100 may include the primary therapy unit 145, the auxiliary therapy unit 150, and the dressing 110. In some embodiments, the auxiliary therapy unit 150 may comprise a rechargeable and disposable canister, as shown in FIG. 8. The primary therapy unit 145 may be, for example, a V.A.C.ULTA™ Therapy Unit available from Kinetic Concepts, Inc. of San Antonio, Texas. In some embodiments, the primary therapy unit 145 may also comprise the container 115 coupled to a side of the primary therapy unit 145. In other embodiments, the container 115 may be housed within the primary therapy unit 145.

In some embodiments, the primary therapy unit 145 may be fluidly coupled to the dressing 110 through the auxiliary therapy unit 150. For example, the primary therapy unit 145 may be fluidly coupled to the dressing 110 via the fluid conductor 410, the second adapter 325, the fluid conductor 335, the auxiliary therapy unit 150, the fluid conductor 330, the first adapter 320, and the fluid conductor 405 to form the negative-pressure pathway. In some embodiments, the primary therapy unit 145 may also be fluidly coupled to the dressing 110 via the fluid conductor 410, the second adapter 325, the third fluid passage 305, the first adapter 320, and the fluid conductor 405 may fluidly couple the primary therapy unit 145 to the dressing 110 to form the sensing pathway. The fluid conductor 405 and the fluid conductor 410 may each be a multi-lumen tube or conduit. The first adapter 320 may fluidly couple at least one lumen of the fluid conductor 405 to the fluid conductor 330 and at least one other lumen of the fluid conductor 405 to the third fluid passage 305. The second adapter 325 may fluidly couple at least one lumen of the fluid conductor 410 to the fluid conductor 335 and at least one other lumen of the fluid conductor 410 to the third fluid passage 305.

In some embodiments, the dressing 110 may include a fluid port, such as a dressing interface 802. The dressing interface 802 may be configured to fluidly couple the fluid conductor 405 to the dressing 110. In some embodiments, the dressing interface 802 may be an elbow connector, which can be coupled to the cover 125 and fluidly coupled to the tissue interface 120. In other embodiments, the dressing interface 802 may be inserted directly through the cover 125 and into the tissue interface 120. In still other embodiments, the fluid conductor 405 may be configured to be inserted through the cover 125 and into the tissue interface 120.

According to an illustrative embodiment, a method for treating a tissue site with negative pressure is further provided. In some embodiments, the method may comprise disposing a dressing adjacent to the tissue site and providing a fluid management device. The fluid management device may comprise a housing, a piston defining a fluid chamber in the housing, a first fluid passage fluidly coupled to the fluid chamber, a first check valve, a second fluid passage fluidly coupled to the fluid chamber, and a second check valve. The first check valve may be configured to prevent egress from the fluid chamber through the first fluid passage and the second check valve may be configured to prevent ingress to the fluid chamber through the second fluid passage. The method may further comprise fluidly coupling the fluid chamber to the dressing though the first check valve, fluidly coupling a negative-pressure source to the fluid chamber through the second check valve, and applying a therapeutic level of negative-pressure from the negative-pressure source to the fluid chamber.

In some embodiments, the method may further comprise separating the negative-pressure source from the fluid chamber. In such embodiments, the second check may close to maintain a therapeutic negative pressure in the fluid chamber and the fluid chamber may receive fluid from the dressing through the first check valve.

The systems, apparatuses, and methods described herein may provide significant advantages. For example, the auxiliary therapy unit 150 may provide a mobile, re-usable, self-emptying, self-charging source of negative pressure that can manage fluid and temporary or transitional negative-pressure treatment.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing 110, the container 115, or both may be separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for managing fluid for negative-pressure treatment of a tissue site, the apparatus comprising:
a fluid reservoir having a variable volume;
a first fluid passage fluidly coupled to the fluid reservoir;
a first check valve configured to prevent egress through the first fluid passage;
a second fluid passage fluidly coupled to the fluid reservoir; and
a second check valve configured to prevent ingress through the second fluid passage.

2. The apparatus of claim 1, wherein the first check valve and the second check valve are configured to open at a therapeutic level of negative pressure.

3. The apparatus of claim 1, wherein the first check valve and the second check valve are configured to open if negative pressure in the fluid reservoir is greater than zero mmHg.

4. The apparatus of claim 1, wherein the first check valve and the second check valve are configured to open if negative pressure in the fluid reservoir is greater than 50 mmHg.

5. The apparatus of claim 1, further comprising: a third fluid passage comprising a first end fluidly coupled to the first fluid passage and a second end fluidly coupled to the second fluid passage.

6. The apparatus of claim 5, wherein:
the first check valve is disposed between the first fluid passage and the first end; and
the second check valve is disposed between the second fluid passage and the second end.

7. The apparatus of claim 5, wherein:
the first fluid passage and the first end of the third fluid passage are configured to be coupled to a tissue interface; and
the second fluid passage and the second end of the third fluid passage are configured to be coupled to a negative-pressure source.

8. The apparatus of claim 1, wherein the fluid reservoir is configured to contract if negative pressure increases in the fluid reservoir and to expand if negative pressure decreases in the fluid reservoir.

9. The apparatus of claim 1, wherein the fluid reservoir is defined by a housing and a piston disposed within the housing.

10. The apparatus of claim 9, further comprising a piston spring coupled to the piston and configured to expand the variable volume of the fluid reservoir if negative pressure in the fluid reservoir decreases.

11. A system for treating a tissue site with negative pressure, the system comprising:
a dressing configured to be disposed adjacent to the tissue site;
a housing and a piston defining a fluid chamber;
a first fluid passage fluidly coupled to the fluid chamber and configured to be fluidly coupled to the dressing;
a first check valve configured to prevent egress from the fluid chamber through the first fluid passage;
a second fluid passage fluidly coupled to the fluid chamber;
a second check valve configured to prevent ingress to the fluid chamber through the second fluid passage; and
a negative-pressure source configured to be coupled to the fluid chamber through the second fluid passage.

12. The system of claim 11, further comprising:
a pressure sensor;
a third fluid passage comprising a first end configured to be fluidly coupled to the dressing and a second end configured to be fluidly coupled to the pressure sensor; and
a controller configured to operate the negative-pressure source based on input from the pressure sensor.

13. The system of claim 11, wherein the fluid chamber is configured to contract if negative pressure increases in the fluid chamber and to expand if negative pressure decreases in the fluid chamber.

14. The system of claim 11, further comprising a piston spring coupled to the piston and configured to expand the fluid chamber if negative pressure in the fluid chamber decreases.

15. The system of claim 11, wherein the second check valve is configured to maintain a therapeutic level of negative pressure in the fluid chamber if the negative-pressure source is separated from the fluid chamber.

16. The system of claim 11, wherein the first check valve and the second check valve are configured to open at a therapeutic level of negative pressure.

17. The system of claim 11, wherein the first check valve and the second check valve are configured to open if upstream negative pressure is greater than zero.

18. The system of claim 11, wherein the first check valve and the second check valve are configured to open if upstream negative pressure is greater than 50 mmHg.

19. A method of treating a tissue site with negative pressure, the method comprising:
disposing a dressing adjacent to the tissue site;
providing a fluid management unit comprising:
a housing,
a piston defining a fluid chamber in the housing,
a first fluid passage fluidly coupled to the fluid chamber,
a first check valve configured to prevent egress from the fluid chamber through the first fluid passage,
a second fluid passage fluidly coupled to the fluid chamber, and
a second check valve configured to prevent ingress to the fluid chamber through the second fluid passage;
fluidly coupling the fluid chamber to the dressing through the first check valve;
fluidly coupling a negative-pressure source to the fluid chamber through the second check valve; and
applying a therapeutic level of negative-pressure from the negative-pressure source to the fluid chamber.

20. The method of claim 19, further comprising separating the negative-pressure source from the fluid chamber, whereby the second check valve closes to maintain a therapeutic level of negative pressure in the fluid chamber and the fluid chamber receives fluid from dressing through the first check valve.

21. (canceled)
